(19) United States
(12) Patent Application Publication
(10) Pub. No.: US 2023/0007971 A1
(43) Pub. Date: Jan. 12, 2023

(54) DUAL STATOR PUMP SPLIT CONNNECTOR

(71) Applicant: HeartWare, Inc., Miami Lakes, FL (US)

(72) Inventors: Andrei George Draghici, Hallandale Beach, FL (US); Kevin R. Seifert, Forest Lake, MN (US); Richard James Lothian, Hollywood, FL (US)

(21) Appl. No.: 17/370,100

(22) Filed: Jul. 8, 2021

(51) Int. Cl. A61M 60/878; H01R 13/631; H01R 13/58

(52) U.S. Cl. CPC A61M 60/878; H01R 13/631; H01R 13/5845

(57) ABSTRACT

A furcated driveline connector configured to couple to an implantable blood pump.

### CROSS-REFERENCE TO RELATED APPLICATION

n/a.

### FIELD

The present technology is generally related to connectors for implantable blood pumps.

### BACKGROUND

Current connectors for implantable blood pumps, which connect a power source to the implantable blood pump, are manufactured within the implantable blood pump which requires high precision assembly to be carried out through most of the process. Moreover, body fluid egress into the driveline through the connector may result in complications.

### SUMMARY

The techniques of this disclosure generally relate to connectors for implantable blood pumps.

In one aspect, the present disclosure provides a furcated driveline connector configured to couple to an implantable blood pump.

In another aspect of this embodiment, the connector further includes a plurality of lumens, and a plurality of conductors disposed within the plurality of lumens.

In another aspect of this embodiment, the connector further includes a first branch opposite a second branch, each branch being the same length.

In another aspect of this embodiment, the connector includes and outer sheath, and wherein the plurality of lumens extends distally away from a distal end of the outer sheath.

In another aspect of this embodiment, wherein the connector further includes a strain relief, and wherein the strain relief includes silicone, and wherein the strain relief is at least one from the group consisting of filled with and over-molded with silicone.

In another aspect of this embodiment, the connector includes a plurality of orientation plugs coupled to a distal end of the plurality of lumens, wherein the orientation plugs are configured to align the plurality of conductors with corresponding conductors within the implantable blood pump.

In another aspect of this embodiment, the orientation plugs include a barb or smooth surface configured to engage the strain relief.

In another aspect of this embodiment, the strain relief increases in diameter as it extends distally from the distal end of the outer sheath.

In another aspect of this embodiment, the strain relief includes a slit extending through a midsection of the strain relief.

In another aspect of this embodiment, each of the orientation plugs extend to a same distal location of the connector.

In one aspect, a furcated driveline connector configured to couple to an implantable blood pump is included, the connector includes a first branch opposite and symmetric with a second branch, each branch including three lumens and a conductor disposed within each lumen.

In another aspect of this embodiment, each of the first branch and the second branch are the same length.

In another aspect of this embodiment, the connector further includes and outer sheath, and wherein the plurality of lumens extends distally away from a distal end of the outer sheath.

In another aspect of this embodiment, the connector further includes a strain relief, and wherein the strain relief includes silicone, and wherein the strain relief is at least one from the group consisting of filled with and over-molded with silicone.

In another aspect of this embodiment, the connector further includes a plurality of orientation plugs coupled to a distal end of the three lumens, wherein the orientation plugs are configured to align the conductors within the implantable blood pump.

In another aspect of this embodiment, the orientation plugs include a barb configured to engage the strain relief.

In another aspect of this embodiment, the strain relief increases in diameter as it extends distally from the distal end of the outer sheath.

In another aspect of this embodiment, the strain relief includes a slit extending through a midsection of the strain relief.

In another aspect of this embodiment, each of the orientation plugs extend to a same distal location of the connector.

In one aspect, a furcated driveline connector configured to couple to an implantable blood pump is included, the connector includes a first branch opposite and symmetric with a second branch, each branch including three lumens and a conductor disposed within each lumen, the connector having a length of between 1-3 inches and a width of 0.1-0.5 inches.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a side inside view of a connector constructed in accordance with the principles of the present application and an exemplary blood pump; and

FIG. 2 is a cross-sectional view of the connector and blood pump shown in FIG. 1.

### DETAILED DESCRIPTION

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

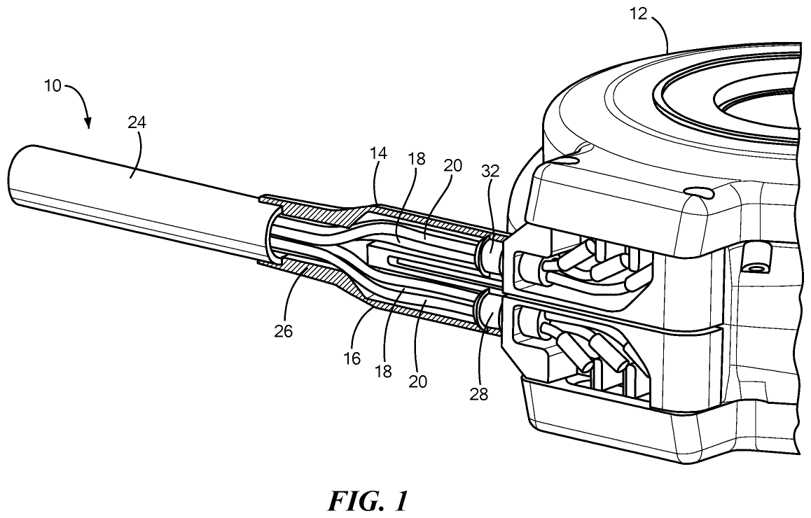

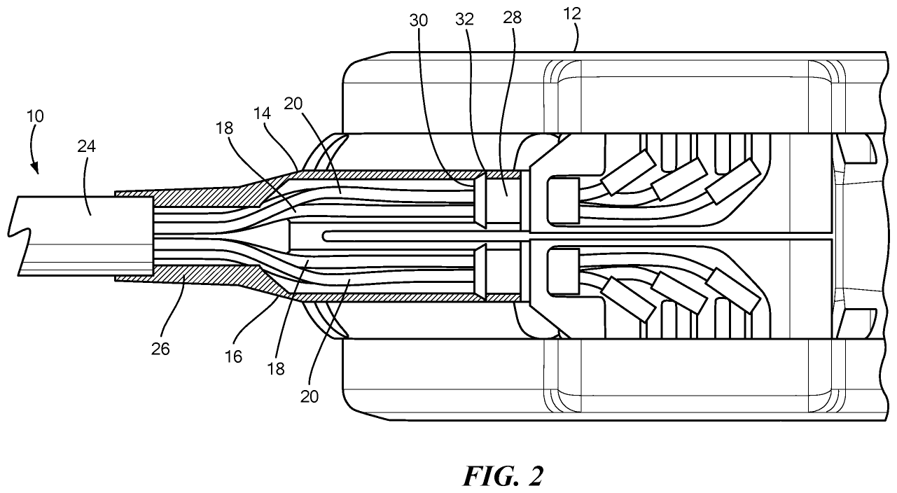

Referring now to the drawings in which like reference designators refer to like elements, there is shown in FIGS. 1-2 an exemplary driveline connector for an implantable blood pump designated generally as “10.” The connector 10 is configured to couple with an implantable blood pump 12. The blood pump 12 may be, without limitation, the HVAD® pump or the MVAD® pump, having a movable element, such as an impeller or a rotor, configured to rotate impel blood from the heart to the rest of the body. The HVAD® Pump is further discussed in U.S. Pat. Nos. 7,997,854 and 8,512,013, the disclosures of which are incorporated herein by reference in the entirety. The MVAD® Pump is further discussed in U.S. Pat. Nos. 8,007,254, 8,419,609, and 9,561,313, the disclosures of which are incorporated herein by reference in the entirety. The connector 10 may further couple with a power source, for example, a battery configured to power the implantable blood pump 12, or with an internal coil configured to received power inductively from the power source.

Continuing to refer to FIGS. 1 and 2, the connector 10 may be furcated or otherwise split to define a first branch 14 opposite and a second branch 16. In other configurations, the connector is a single branch 14 without being furcated. For example, the first branch 14 and the second branch 16 may be symmetric and have the same length. Each of the first branch 14 and the second branch 16 may each include a plurality of lumens 18 configured to receive a corresponding conductor 20. The conductors 20 are configured to electrically engage with the implantable blood pump 12 to provide power to the pump 12. In an exemplary configuration, three conductors 20 are disposed in each of the first branch 14 and the second branch 16 for a total of 6 conductors each within a corresponding one of the plurality of lumens 18. In one configuration, an outer sheath 24 is included surrounding a portion of the connector 10. For example, as shown in FIGS. 1 and 2, the first branch 14 and the second branch 16 and the plurality of lumens 18 therein extend away from a distal end of the outer sheath 24. A strain relief 26 is included and encloses at least a portion of the first branch 14 and the second branch 16. The strain relief 26 is either filled with or over-molded with silicone to provide flexibility to the connector 10. A slit is provided between the first branch 14 and the second branch 16 in the strain relief 26 to separate both branches at the mid-section. In other configurations, the first branch 14 and the second branch 16 are interlocked or otherwise connected to prevent tissue ingrowth between the branches. In an exemplary configuration, the strain relief 26 increases in diameter from a position proximate the distal end of the outer sheath 24 to a position wherein the first branch 14 and the second branch 16 split.

Continuing to refer to FIGS. 1-2, the connector 10 includes a plurality of orientation plugs 28 coupled to a distal end of the plurality of lumens 18. The orientation plugs 28 are configured to align the conductors 20 within the implantable blood pump 12. For example, the orientation plugs 28 may include a plurality of channels through which each conductor 20 is fed through to align with implantable blood pump 12. In one configuration, an entirety of the orientation plugs 28 is disposed within the strain relief 26. A proximal end 30 of the orientation plugs may also include an anchoring flange or barb 32 which is configured to anchor on to the strain relief 26. The orientation plugs 28 may be secured to a header of the pump 12 or may be screwed into the header. In one configuration, the orientation plugs 28 are aligned such that the distal ends of each orientation plug 28 terminate at the same location.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A furcated driveline connector configured to couple to an implantable blood pump.

2. The connector of claim 1, further including a plurality of lumens, and a plurality of conductors disposed within the plurality of lumens.

3. The connector of claim 2, wherein the connector includes a first branch opposite a second branch, each branch being the same length.

4. The connector of claim 3, wherein the connector includes and outer sheath, and wherein the plurality of lumens extends distally away from a distal end of the outer sheath.

5. The connector of claim 4, further including a strain relief, and wherein the strain relief includes silicone, and wherein the strain relief is at least one from the group consisting of filled with and over-molded with silicone.

6. The connector of claim 5, wherein the connector includes a plurality of orientation plugs coupled to a distal end of the plurality of lumens, wherein the orientation plugs are configured to align the plurality of conductors with the implantable blood pump.

7. The connector of claim 6, wherein the orientation plugs include a barb configured to engage the strain relief.

8. The connector of claim 7, wherein the strain relief increases in diameter as it extends distally from the distal end of the outer sheath.

9. The connector of claim 8, wherein the strain relief includes a slit extending through a midsection of the strain relief.

10. The connecter of claim 9, wherein each of the orientation plugs extend to a same distal location of the connector.

11. A furcated driveline connector configured to couple to an implantable blood pump, the connector including a first branch opposite and symmetric with a second branch, each branch including three lumens and a conductor disposed within each lumen.

12. The connector of claim 11, wherein each of the first branch and the second branch are the same length.

13. The connector of claim 12, wherein the connector includes and outer sheath, and wherein the plurality of lumens extends distally away from a distal end of the outer sheath.

14. The connector of claim 13, further including a strain relief, and wherein the strain relief includes silicone, and wherein the strain relief is at least one from the group consisting of filled with and over-molded with silicone.

15. The connector of claim 14, wherein the connector includes a plurality of orientation plugs coupled to a distal end of the three lumens, wherein the orientation plugs are configured to align the conductors within the implantable blood pump.

16. The connector of claim 15, wherein the orientation plugs include a barb configured to engage the strain relief.

17. The connector of claim 16, wherein the strain relief increases in diameter as it extends distally from the distal end of the outer sheath.

18. The connector of claim 17, wherein the strain relief includes a slit extending through a midsection of the strain relief.

19. The connecter of claim 18, wherein each of the orientation plugs extend to a same distal location of the connector.

20. A furcated driveline connector configured to couple to an implantable blood pump, the connector including a first branch opposite and symmetric with a second branch, each branch including three lumens and a conductor disposed within each lumen, the connector having a length of between 1-3 inches and a width of 0.1-0.5 inches.
